(19) United States
(12) Patent Application Publication
(10) Pub. No.: US 2004/0198663 A1
(43) Pub. Date: Oct. 7, 2004

(54) Method of treating cardiac ischemia by using erythropoietin

(76) Inventors: John E. Baker, Wauwatosa, WI (US); Yang Shi, Wauwatosa, WI (US)

Correspondence Address: WHYTE HIRSCHBOECK DUDEK S C, 555 EAST WELLS STREET, SUITE 1900, MILWAUKEE, WI, 53202, US

(21) Appl. No.: 10/817,058

(22) Filed: Apr. 2, 2004

Related U.S. Application Data
(60) Provisional application No. 60/460,684, filed on Apr. 4, 2003.

(51) Int. Cl. A61K 38/18

(52) U.S. Cl. 514/12

(57) ABSTRACT

Methods of substantially immediately protecting and increasing the resistance of the heart against the effects of ischemia, and pharmaceutical compositions that incorporate erythropoietin for use in such methods are disclosed. The methods of the invention are useful in treating or reducing damage to the heart from the incidence of ischemia.

### CROSS-REFERENCE TO RELATED APPLICATION

This application claims the benefit of U.S. Provisional application Serial No. 60/460,684, filed on Apr. 4, 2003, the teachings and disclosures of which are herein incorporated by reference.

### STATEMENT REGARDING FEDERALLY SPONSORED RESEARCH

This invention was made with United States government support from the National Institutes of Health (NIH), National Heart and Lung Institute (NHLI), NIH/NHLI Grant No. HL54075. The United States government has certain rights in this invention.

### FIELD OF THE INVENTION

The present invention relates generally to methods and products for use in treating myocardial ischemia, and more particularly to the application of erythropoietin to increase resistance to myocardial ischemia and products that incorporate erythropoietin for such use.

### BACKGROUND OF THE INVENTION

Congenital heart defects occur in one out of every 125 newborn children (J. Hoffman, in J. Mooer and J. Hoffman (eds.), Pediatric Cardiovaxcular Medicine, Churchill Livingstone, N.Y., pp. 257-262 (2000). One third of these children require a major surgical procedure within the first year of life to prevent premature death. Many of these children exhibit varying degrees of cyanosis where the myocardium is chronically perfused with hypoxic blood and treatments for protecting the hearts of these children during corrective surgery would be useful. However, the mechanisms by which cyanotic congenital heart defects modify the myocardium are not clearly understood.

An animal model in which rabbits are raised from birth in a hypoxic environment has been developed to investigate the effects of chronic hypoxia on signal transduction mechanisms. (Baker et al., Am. J. Physiol. 268:H1165-1173 (1995)). It has been shown that infant human and rabbit hearts adapt to chronic hypoxemia by activation of PKC_{ε}, p38 MAP kinase and JUN kinase signaling pathways and by increasing nitric oxide production. (Rafiee, P. et al., Circulation 106:239-45 (2002); Shi, Y. et al., Free Radic. Biol. Med. 29:695-703 (2000)). Activation of these protein kinase-signaling pathways and nitric oxide synthase in infant hearts adapted to chronic hypoxia is associated with increased resistance to ischemia, which is mediated by ATP-sensitive K⁺ (K_{ATP}) channels. (Baker et al., J. Mol. Cell Cardiol. 29:845-848 (1997), Baker et al. Circ 95:1278-1285 (1997)).

Chronic hypoxia from birth also results in erythropoiesis as manifested by an increase in hemoglobin and hematocrit. (Baker et al., Am J Physiol 268:H1165-1173 (1995)). Erythropoietin activates p38 MAP kinase and JUN kinase signaling pathways and can increase resistance to cerebral ischemia. (Nagata et al., Blood 92:1859-69. (1998); Siren et al., Proc Natl Acad Sci USA 98:4044-9 (2001)).

Ischemia is a condition resulting from a decrease or lack of blood flow and oxygen to a part of the body such as the heart (cardiac ischemia; ischemic cardiomyopathy), which causes damage to tissue that is distal to a blockage. During certain surgical procedures such as cardiac surgery and organ transplantation, the flow of blood is stopped temporarily and then resumed (reperfusion), resulting in ischemia-reperfusion injury. During a heart attack, the blood that supplies the heart is stopped, also resulting in ischemic injury.

It would be desirable to provide a therapy and therapeutic products to effectively increase resistance of the heart to ischemia, including in the setting of cardiac surgery (global myocardial ischemia) and heart attack (regional myocardial ischemia).

### SUMMARY OF THE INVENTION

The present invention provides methods of protecting mammalian tissue and organs, particularly the heart, from the effects of ischemia, and pharmaceutical compositions that incorporate erythropoietin (EPO) for use in such methods.

It has been found that the administration of erythropoietin to a mammal (“patient”) according to the methods of the invention provides beneficial immediate cardioprotective effects on the heart, particularly in increasing the resistance of the heart to ischemia. According to the invention, erythropoietin is administered as a therapeutic agent for cardioprotection and in the treatment of ischemia, including injuries caused by ischemia-reperfusion effects.

The invention provides methods of immediately reducing the effects of myocardial ischemia in a human or other mammal (“patient”) to prevent or decrease damage to the heart. The method involves administering erythropoietin in a pharmaceutical composition in an amount effect to reduce the damaging effects of myocardial ischemia.

In one embodiment, the method comprises preconditioning a patient against myocardial ischemia (ischemic injury) by administering erythropoietin to a patient at a concentration and duration effective to prevent or reduce such injury substantially immediately upon its occurrence. In another embodiment, the method involves administering erythropoietin to a patient prior to a scheduled or planned ischemic event such as a surgical procedure, to precondition the patient. Preferably, a composition containing an effective amount of EPO to result in a blood level of about 0.5-10.0 U/ml EPO within a short time of administration of the EPO composition, preferably within about 1-20 minutes, is administered to the patient prior to an ischemic event, generally about 1-60 minutes or longer, preferably about 5-15 minutes prior to the event. A preferred dosage amount is about 50-5,000 U/kg of EPO.

In another embodiment of a method according to the invention, a donor organ (e.g., heart) can be administered erythropoietin prior to transplantation via the vascular system at a concentration and duration effective to prevent or reduce injury from the effects of ischemia and reperfusion from the transplantation procedure. Preferably, a solution containing an effective amount of EPO, preferably a concentration of about 0.5-10.0 U/ml EPO, is administered to the organ prior to transplantation for a period of about 1-60 minutes or longer, preferably for about 5-20 minutes to provide a concentration of about 0.5-10.0 U/ml EPO within the organ.

In another embodiment, the erythropoietin can be administered at the commencement of and/or subsequent to an ischemic event for treating, preventing or decreasing injury to the heart. Examples of such events include a surgical procedure during which an ischemia-reperfusion injury can occur upon the reperfusion of an organ or tissue such as heart or other organ surgery, a transplant procedure, and the like. In addition, a patient experiencing symptoms of a disease state such as a myocardial infarction, for example, can be administered erythropoietin to substantially immediately decrease ischemic injury to the heart. The erythropoietin can be administered to a patient in a pharmaceutical composition containing a therapeutic amount of EPO effective to substantially immediately decrease or prevent damage to the heart caused by the ischemic event. Preferably, a composition containing an effective amount of EPO to result in a blood level of about 0.5-10.0 U/ml EPO is administered to the patient at or about the commencement of the ischemic event and/or within a short time subsequent to the ischemic event for an effective duration, to result in substantially immediate cardioprotection and decreased ischemic injury, preferably within about 1-20 minutes of administration. A preferred dose amount is about 50-5,000 U/kg of EPO.

While not meant to limit the invention, it is believed that one way that erythropoietin can reduce the injury caused by ischemia and provide a substantially immediate cardioprotective effect is by activating potassium channels and protein kinases. Accordingly, the invention also provides a method of activating a cardioprotective signaling pathway, for example, to activate a protein kinase (e.g., MAP kinase) or a potassium channel (e.g., K_{ATP}) to provide a cardioprotective effect. Preferably, a composition containing an effective amount of EPO to result in a blood level of about 0.5-10.0 U/ml EPO substantially immediately after administration, preferably within about 1-20 minutes, with a preferred dose amount being about 50-5,000 U/kg of EPO.

The invention further provides pharmaceutical compositions comprising erythropoietin in a physiologically-acceptable carrier. The compositions are formulated to provide an effective amount of erythropoietin to provide a substantially immediate cardioprotective effect, for example, to decrease the effects of ischemia on the heart and/or other tissue or organ, preferably at an EPO concentration to result in a blood level of about 0.5-10.0 U/ml, preferably at or about 1 U/ml, preferably within about 1-20 minutes of administration. A preferred pharmaceutical composition is formulated to provide a dosage amount of about 50-5,000 U/kg of EPO.

The methods of the invention advantageously provide a substantially immediate cardioprotective effect against injury caused by ischemia. Previously known procedures for treating myocardial ischemia involve administering EPO and a subsequent waiting period or interval of 8 to 24 hours before a cardioprotective effect takes place. However, when presented with symptoms of heart attack, stroke or other disease state, or in conducting an organ transplant, for example, immediate cardioprotection or cerebroprotection against ischemic injury is desired rather than a delayed effect. The invention eliminates or substantially reduces the waiting period for cardioprotection to take effect.

### BRIEF DESCRIPTION OF THE DRAWINGS

FIG. 1 is a depiction of an experimental protocol used for the erythropoietin concentration response studies.

FIG. 2 is a graphic depiction of the results of erythropoietin concentration-response study illustrating the percent (%) recovery of left (▪) and right (□) ventricular developed pressure in the heart following 15 minutes of treatment with erythropoietin at 0.5, 1.0, 2.5, 5.0, and 10.0 U/ml prior to a 30 minute global ischemia and a 35 minute reperfusion. Data are means±SD, n=8 hearts/group. *=P<0.05, EPO vs. drug-free control.

FIG. 3 is a graphic depiction of the results of protein kinase-mediated cardioprotective effects of erythropoietin in infant rabbit hearts. Recovery of left ventricular developed pressure (▪) following 15 minutes of treatment with erythropoietin (at 1.0 U/ml) and protein kinase inhibitors prior to a 30 minute ischemia and a 35 minute reperfusion. The protein kinase inhibitors included chelerythrine, SB203580, PD98059, and SP600125. Data are means±SD (n=8 hearts/group). *=P<0.05, EPO vs. drug free control. +=P<0.05, EPO+drug vs. EPO.

FIG. 4 is a Western blot of total cell lysates, cytosolic (“cyto”) and particulate (“part”) fractions of hearts treated with 1.0 U/ml erythropoietin for 5 minutes or 15 minutes to demonstrate cardioprotection by erythropoietin-involvement of protein kinases. Data are representative of three blots for each antibody. (PKCε=protein kinase C; p38 MAPK and p44/42 MAPK=mitogen-activated protein kinases; JNK=Jun N-terminal kinase)

FIG. 5 is a graphic depiction of the results of potassium channel mediated cardioprotective effects of erythropoietin. Recovery of left ventricular developed pressure (▪) following a 15 minute treatment with erythropoietin (1.0 U/ml) and potassium channel blockers prior to a 30 minute ischemia and 35 minute reperfusion. Potassium channel blockers were glibenclamide (“Glib”) at 3 μM, HMR 1098 at 30 μM, 5-HD at 300 μM, and paxilline at 1 μM. Data are means±SD (n=8 hearts/group). *=P<0.05, EPO vs drug free control. +=P<0.05, EPO+drug vs EPO.

FIG. 6 is a graphic depiction of the results of the role of nitric oxide synthase in erythropoietin-induced cardioprotection. Recovery of left ventricular developed pressure (▪) following a 15 minute treatment with erythropoietin (1.0 U/ml) and nitric oxide synthase inhibitors prior to a 30 minute ischemia and 35 minute reperfusion. Nitric oxide synthase inhibitors included L-NAME at 200 μM and L-NMA at 100 μM. Data are means±SD (n=8 hearts/group). *=P<0.05, EPO vs. drug free control.

FIG. 7 is a graphic depiction of the results of cardioprotection by erythropoietin in chronically hypoxic hearts. Recovery of left and right ventricular developed pressure following a 15 minute treatment with erythropoietin (1.0 U/ml) prior to a 30 minute ischemia and 35 minute reperfusion. Data are means±SD (n=8 hearts/group). Control=□; EPO treated=▪.

FIG. 8 is a schematic representation of signaling pathways by which erythropoietin may confer immediate cardioprotection.

FIG. 9 is a graphic depiction of the effect of erythropoietin (1.0 U/ml) on myocardial infarct size (as percentage % of the heart) when administered 15 minutes prior to a 30 minute regional myocardial ischemia induced by suture ligation of the left main coronary artery and 3 hours reperfusion. *=P<0.05, with EPO vs. without EPO (control).

FIG. 10 is a graphic depiction of the effect of erythropoietin (1.0 U/ml) to increase post-ischemic recovery of left ventricular developed pressure when administered 15 minutes prior to a 30 minute myocardial ischemia induced by suture ligation of the left main coronary artery and 3 hours reperfusion. *=P<0.05, with EPO vs. without EPO (control).

### DETAILED DESCRIPTION OF THE PREFERRED EMBODIMENTS

The invention is directed to methods of using erythropoietin to substantially immediately protect the heart of a patient against injury caused by myocardial ischemia.

By “immediately” or “substantially immediately”, it is meant that the cardioprotective effect against ischemia occurs instantaneously or within a short time period following administration of a composition comprising erythropoietin, preferably within at least about 35 minutes following administration, preferably within about 1-20 minutes, preferably within about 1-15 minutes, preferably within about 1-10 minutes, and preferably within about 1-5 minutes.

Erythropoietin

Erythropoietin (EPO) is a glycoprotein hormone produced by the kidneys in response to the oxygen concentration in the blood. Normal blood EPO values range from about 0 to 19 milliunits per milliliter (mU/ml). EPO acts on the bone marrow to increase the production of red blood cells. A normal hemocrit (% blood that is occupied by red blood cells) is normally between 40% and 52% for men, and between 35% and 46% for women. Lower values are indicative of anemia. An increase in EPO blood levels primarily occurs in response to tissue hypoxia from decreased blood oxygen caused by anemia, for example. The link between reduced EPO blood levels and heart disease is not conclusive.

Suitable EPO preparations for use in the methods of the invention include naturally occurring EPO (e.g., EPO extracted from human urine and purified) or recombinant human EPO (rhEPO), and modifications thereof having substantially comparable physiological and biological properties to that of mammalian, especially human EPO and rhEPO. EPO can be obtained, for example, as described in U.S. Pat. No. 5,661,125 (Strickland) and U.S. Pat. No. 5,955,422 (Lin, Kirin-Amgen, Inc., Thousand Oaks, Calif.). Recombinant human erythropoietin (rhEPO) is commercially available as EPOGEN® (Epoietin alpha) (Amgen Inc., Thousand Oaks, Calif.) and as PROCRIT® (Ortho Biotech Inc., Raritan, N.J.). Various modified forms of erythropoietin are also encompassed by the present invention with activities directed towards improving the erythropoietic activity of the molecule including, but are not limited to, for example, those with amino acids at the carboxy terminus described in U.S. Pat. No. 5,457,089 and in U.S. Pat. No. 4,835,260; erythropoietin isoforms with various numbers of sialic acid residues per molecule, such as described in U.S. Pat. No. 5,856,292; polypeptides described in U.S. Pat. No. 4,703,008; agonists described in U.S. Pat. No. 5,767,078; peptides which bind to the erythropoietin receptor as described in U.S. Pat. Nos. 5,773,569 and 5,830,851, small-molecule mimetics as described in U.S. Pat. No. 5,835,382; erythropoietin modified with polyethylene glycol as described in U.S. Pat. No. 6,586,398, and erythropoietin analogs described in WO 9505465, WO 9718318 and WO 9818926. Additional modifications may include but are not limited to, for example, carbamylated erythropoietins, succinylated erythropoietins, acetylated erythropoietins, biotinylated erythropoietins, iodinated erythropoietins, and carboxymethyllysyl erythropoietins, and the like.

Pharmaceutical Compositions

EPO is formulated in a pharmaceutical composition by combining the EPO with a pharmaceutically acceptable carrier in a therapeutic amount effective to reduce myocardial ischemia in a patient to decrease damage to the heart.

The pharmaceutical composition can be administered intravenously, subcutaneously, intramuscularly, intraperitoneally, transdermally, nasally, or by suppository. In general, systemic administration is preferable.

Erythropoietin as the active ingredient for the reduction of myocardial ischemia can be formulated with conventional pharmaceutically acceptable parenteral carriers for administration by injection, which are compatible with EPO, essentially nontoxic and non-therapeutic such as sterile distilled water, saline, Ringer's solution, dextrose solution, Hank's solution, or the like, and physiologically acceptable to the patient. For parenteral administration, the EPO can be incorporated into a solution or suspension, preferably a buffered solution or suspension.

An intranasal formulation can be prepared as a parenteral preparation as a solution or suspension for delivery in the form of drops or spray using, for example, a nebulizer or atomizer for inhalation by the patient. A parenteral or intranasal preparation can be aseptically enclosed in ampoules, vials, disposable syringes, and other suitable containers.

In a transdermal delivery system, the EPO can be prepared as a topical composition in a liquid or semi-liquid form such as a lotion, cream, ointment, gel, paste, solution or suspension. Transdermal delivery of EPO by skin penetration can be enhanced by use of occlusive techniques (e.g., wrap or impermeable plastic film) that hydrate the skin and increase skin temperature, or by the use of a suitable penetrating agent (e.g., water, polyols such as glycerin and propylene glycol).

A suppository dosage form can be prepared by combining the EPO with a carrier comprising a cocoa butter base, or a water-soluble or dispersible base such as polyethylene glycols and glycerides, that is solid at room temperature (about 20° C.) and melts at body temperature. Suppositories are typically individually foil wrapped, or hermetically sealed in a molded plastic container.

Patient treatment using the method of the present invention involves administering therapeutic amounts of the EPO pharmaceutical composition, which contains EPO in an amount effective to provide a suitable dosage for its intended purpose.

The activity (in units) of erythropoietin and erythropoietin-like molecules is traditionally defined based on its effectiveness in stimulating red cell production in rodent models (and as derived by international standards of erythropoietin). One unit (U) of regular erythropoietin (MW of ˜34,000) is about 10 ng of protein (1 mg protein is approximately 1000,000 U). Thus a dose of 50 U/kg would be equivalent to 500 ng/kg or 0.5 mg/kg.

Preferred compositions and preparations are prepared so that a dosage unit form contains an amount of EPO effective to provide a blood concentration of about 0.5-10.0 U/ml EPO, preferably about 0.5-5.0 U/ml EPO, preferably about 0.5-2.0 U/ml EPO, preferably about 0.8-1.5 U/ml EPO, and preferably about 1.0 U/ml EPO, immediately or substantially immediately after administration. Preferably, the composition contains EPO in an amount effect to provide a desired EPO blood concentration within at least about 35 minutes following administration, preferably within about 1-20 minutes, preferably within about 1-15 minutes, preferably within about 1-10 minutes, and preferably within about 1-5 minutes. A preferred dose amount is about 50-5,000 U (units) EPO/kg body weight, which can be adjusted to provide the optimum therapeutic response. The effective dose amount of EPO that is administered can vary depending on the route of administration, and the age, weight and/or health of the patient, and other factors such as the condition being treated.

The pharmaceutical compositions can include small amounts of adjuvants such as buffers and preservatives to maintain isotonicity, physiological and pH stability, which do not adversely affect the efficacy of the EPO composition.

The composition can be administered in a single dose, in multiple doses, or continuously for a desired period of time. The amount administered is that amount effective to achieve the desired effect as described above. The amount is preferably that amount that prevents or reduces myocardial ischemia in a patient. Preferably, the amount that is administered is effective to increase the blood level of EPO in a patient to about 100 times, preferably to about 500 times, above normal EPO blood levels, or an EPO blood level of about 100-5000 mU/ml, preferably about 3000 mU/ml, preferably about 1000 mU/ml. This amount can be determined by testing the patient's blood.

In a preferred embodiment, a patient is administered a single treatment (rather than multiple or repeated treatments daily, for example) of about 50-5,000 U/kg EPO to confer a substantially immediate cardioprotective effect.

Methods

The methods of the invention utilize erythropoietin to protect the heart of a patient against injury caused by myocardial ischemia. The methods involve administering a pharmaceutical composition comprising EPO to a human or other mammal in an amount effective to achieve the desired effect in treating myocardial injury caused by ischemic incidences.

The duration of administration of the EPO composition generally depends on the formulation of the EPO composition and the desired dose amount to be administered. Other factors that can vary the time period of administration include, for example, the type of treatment being provided or procedure being conducted, for example, preparation of an organ to be transplanted, preconditioning of a transplant (donor) organ, treatment of a heart attack or stroke patient, treatment prior to, during and after a heart surgery, prevention of a ischemia-reperfusion injury, etc.; and the desired or required duration of the treatment or procedure being conducted; among other factors.

For the benefits of substantially immediate cardioprotection against ischemic injury by the methods of the invention, it is preferred that the EPO composition is administered for a period of about 1-60 minutes, preferably up to about 30 minutes, preferably up to about 20 minutes, preferably about 5-15 minutes. The duration of the administration can be extended as needed, for example up to 24 hours or longer, as needed to confer cardioprotection and/or provide additional therapeutic effects without significantly increasing the patient's normal hemoglobin concentration or hematocrit level (i.e., less than about 10% increase).

In an embodiment of the method, a pharmaceutical composition containing EPO in an amount effective to reduce an ischemic event is administered to a patent prior to the ischemic event, for example, prior to a scheduled surgical procedure, to precondition the patient against ischemic injury. For example, surgical procedures that can lead to ischemic injury include heart surgery, a heart transplantation procedure, angioplasty, laparoscopic surgery, and the like. As another example, EPO can also be beneficially administered to a donor patient for preservation of a donor organ for transplantation (e.g., a heart transplant) and prevention of ischemic-reperfusion injury to the organ. Preferably, the EPO composition is administered to a patient prior to an ischemic event to provide a blood concentration of the EPO for substantially immediate cardioprotection, preferably to provide a blood level of about 0.5-10.0 U/ml EPO within an about 1-35 minute period. The EPO composition is preferably administered prior to the event for a time period (duration) of about 1-60 minutes, preferably about 1-30 minutes, preferably about 1-20 minutes, preferably for a period of about 5-15 minutes.

As a further example, to reduce the effects of myocardial ischemia in an organ transplant recipient, an organ to be transplanted such as a heart, for example, can be exposed to an effective amount of erythropoietin in a pharmaceutically acceptable formulation to reduce the effects of ischemia and reperfusion on the organ upon transplantation. The transplant organ can be exposed to the erythropoietin, for example, by infusing via the vasculature, a solution containing an effective amount of erythropoietin to the organ to be transplanted. Preferably, the infusion of EPO to the organ provides a blood EPO concentration of about 0.5-1.0 U/ml EPO within an about 1-35 minute period. The exposure of the transplant organ to erythropoietin can be continuous for the period preceding transplantation, and is preferably for about 1-60 minutes, preferably about 1-30 minutes prior to transplantation, preferably for a period of about 5-15 minutes.

Another method of the invention involves administering EPO in a therapeutic amount effective to substantially immediately treat, prevent or decrease ischemic injury to the heart at or after the onset of an ischemic event, for example, during a surgical procedure or upon experiencing symptoms of a disease state to reduce the severity of a myocardial ischemic incident and prevent further damage. Examples of surgical procedures that can lead to ischemic injury, particularly ischemic-reperfusion injury, include heart surgery, a heart transplantation procedure, angioplasty, laparoscopic surgery, and the like. For example, EPO can be administered to a patient during a heart surgery to decrease damage caused by ischemia and reperfusion during the procedure. As another example, EPO can be administered at the commencement of reperfusion, during reperfusion, or both. Examples of disease states for which the method can be applied to provide substantially immediate cardioprotection against ischemic injury to the heart upon presentation of symptoms include, for example, myocardial infarctions, pulmonary infarctions, peripheral vascular occlusive disease, stroke, cerebral infarction, vascular occlusion, pre-natal or post-natal oxygen deprivation, trauma, including surgery and radiotherapy, chronic obstructive pulmonary disease, emphysema, adult respiratory distress syndrome, septic shock, sickle cell crisis, dysrhythmias, nitrogen narcosis and neurological deficits caused by heart-lung bypass procedures, and the like. Preferably, the EPO composition is administered to the patient at the commencement of the ischemic event and/or within a short time period subsequent to the commencement of the ischemic event to provide a blood EPO concentration of about 0.5-1.0 U/ml EPO within an about 1-35 minute period. The duration of administration of EPO to the patient can be for an effective time period, preferably for about 1-60 minutes, preferably for about 1-30 minutes, preferably for a period of about 5-15 minutes.

Yet another method of the invention involves administering EPO in an amount effective to activate a cardioprotective signaling pathway. In one embodiment, the method comprises administering erythropoietin in a pharmaceutical composition in an amount and duration effective to activate a protein kinase to provide a substantially immediate cardioprotective effect against ischemic injury, preferably a composition to achieve a blood level of about 0.5-10.0 U/ml of EPO when delivered to a patient (human or other mammal) over an about 1-35 minute period. The duration of administration of EPO to the patient can be for an effective time period, preferably for about 1-60 minutes, preferably for about 1-30 minutes, preferably for a period of about 5-15 minutes. Examples of protein kinases that can be activated according to the invention include protein kinase C (PKC_{ε}), mitogen-activated protein (MAP) kinases such as p38 MAPK and p42/44 MAPK, and Jun N-terminal kinase (JNK).

In another embodiment, erythropoietin is administered in a pharmaceutical composition in an effective amount and duration to activate a potassium channel such as K_{ATP} and KCa, to achieve substantially immediate cardioprotection against ischemic injury, preferably to achieve a blood level of about 0.5-10.0 U/ml of EPO when delivered to a patient over an about 1-35 minute period. The EPO composition is administered to the patient for an effective time period, preferably for about 1-60 minutes, preferably for about 1-30 minutes, preferably for a period of about 5-15 minutes to provide a substantially immediate cardioprotective effect against ischemic injury.

Myocardial ischemic injuries that can be prevented or reduced according to the invention include coronary artery disease, myocardial infarction, coronary heart disease, Prinzmetal angina, cardiac rupture and congestive heart failure, for example. Efficacy of the composition and its administration can be monitored by the absence or a decrease in severity of a myocardial ischemic injury by using standard methodology such as cardiac enzyme leakage, cardiac contractile protein leakage, left and right cardiac ventricular cavity pressures, arrhythmias and S-T segment elevation. The effect of the EPO composition can be evaluated about 1-48 hours after administration of the pharmaceutical composition.

The invention will be further described with reference to the following detailed examples, wherein methodologies are described below. These examples are not meant to limit the scope of the invention that has been set forth in the foregoing description. It should be understood that variations and modifications within the concepts of the invention can be made while remaining within the spirit and scope of the invention. The disclosure of cited references, patents, and patent applications throughout the application are incorporated by reference herein.

### EXAMPLE 1

### Immediate Cardioprotective Effects of Erythropoietin Against Global Ischemia and Mediation by Activation of Protein Kinases and Potassium Channels

To determine a possible role for erythropoietin in cardioprotection and the underlying mechanisms, infant rabbit hearts were treated with human recombinant erythropoietin prior to ischemia. The objectives of the study were to determine whether acute exposure (versus chronic exposure) of the heart to erythropoietin would increase resistance to subsequent ischemia, the erythropoietin concentration that confers optimal protection of the heart, the involvement and cellular location of protein kinase signaling pathways, and the role of potassium channels and nitric oxide synthase in mediating cardioprotection.

The study was directed to determining whether erythropoietin (0.5-10.0 U/ml) confers immediate cardioprotection in infant rabbit hearts and the contribution of protein kinases, nitric oxide synthase and potassium channels to the underlying mechanism. Hearts from normoxic infant Zealand White rabbits (n=8/group) were isolated and perfused in the Langendorff mode. Biventricular function was recorded under steady-state conditions prior to 30 minutes global no flow ischemia and 35 minutes reperfusion.

Methods

Animals. Rabbits used in the study received humane care in compliance with the “Guide for the Care and Use of Laboratory Animals” formulated by the National Research Council, 1996. Infant New Zealand White rabbits were maintained for 10 days in a normoxic (SaO2>95%) or hypoxic (SaO2<85%) environment as described in Baker et al., Circulation 99:1249-54 (1999).

Reagents. Recombinant human erythropoietin was obtained from Cell Science, Inc. (Norwood, Mass.). Glibenclamide was obtained from Calbiochem (San Diego, Calif.). 5-HD was purchased from Sigma-Aldrich (St. Louis, Mo.) with HMR 1098 kindly provided by Dr. Garrett Gross. Chelerythrine, SP600125, PD98059 and SB203580 were obtained from Sigma-Aldrich (St. Louis, Mo.), Biomol Research Laboratories, Inc. (Plymouth Meeting, Pa.), and Calbiochem (San Diego, Calif.). Paxilline was obtained from Biomol Research Laboratories, Inc. (Plymouth Meeting, Pa.). Antibodies to phosphorylated and nonphosphorylated p44/42 MAP kinase and p38 MAP kinase were obtained from Cell Signaling Tech (Beverly, Mass.). Anti PCK_{ε} was obtained from Calbiochem (San Diego, Calif.) and anti phospho PCK_{ε} was obtained from Upstate Biotech, Inc. (Lake Placid, N.Y.). The secondary antibody was horseradish peroxidase obtained from Zymed (South San Francisco, Calif.).

Isolated heart perfusion. Isolated rabbit hearts were perfused with bicarbonate buffer at constant pressure in a retrograde manner and instrumented as described in Baker et al., Circulation 99:1249-54 (1999). Protein kinase inhibitors, potassium channel blockers or nitric oxide synthase inhibitors were added to this perfusate as needed. A 3-way tap, located immediately above the site of cannulation, allowed the entire perfusate to be diverted away from the heart to produce global, no-flow ischemia. Reperfusion was achieved by repositioning of the tap to allow perfusate to be delivered to the heart. Left and right ventricular function was monitored continuously throughout each experiment as described in Baker et al., Circulation 99:1249-54 (1999). End-diastolic pressure was initially set to 3 mmHg for 2 minutes. The balloons were then progressively inflated with a microsyringe to set end-diastolic pressures to 8 mmHg for the left ventricle and 4 mmHg for the right ventricle, with developed pressure and heart rate recorded during steady-state conditions. Coronary flow rate was measured throughout the experiment by timed collections of the coronary effluent from the right side of the heart into a graduated cylinder. Coronary flow rate was expressed as milliliters per minute per gram wet weight.

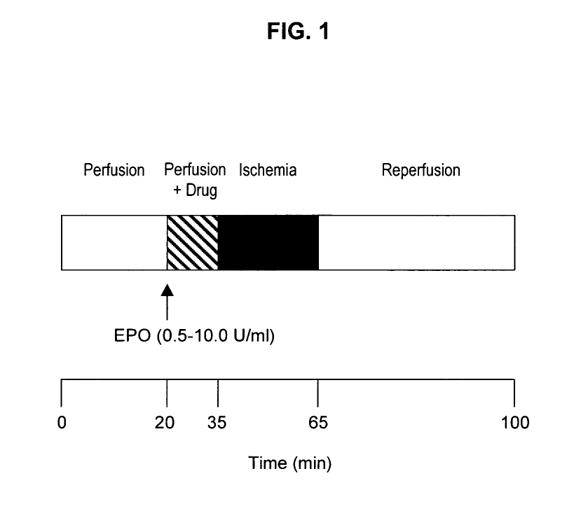

Resistance to myocardial ischemia. Hearts from infant rabbits were perfused with bicarbonate buffer, and biventricular function was monitored continuously throughout each experiment as described in Rafiee et al., Circulation 106:239-45 (2002). For concentration response studies, hearts were then perfused with erythropoietin (0.5-10.0 U/ml) for 15 minutes prior to 30 minutes ischemia and 35 minutes reperfusion. The experimental protocol used is shown in FIG. 1. For mechanism studies with protein kinase inhibitors, potassium channel blockers or nitric oxide synthase inhibitors, hearts were perfused with drugs for 15 minutes alone followed by 15 minutes in combination with erythropoietin prior to ischemia. Hearts perfused with protein kinase inhibitors or potassium channel blockers alone in the absence of erythropoietin for 30 minutes prior to ischemia served as untreated controls for these studies. Recovery of post-ischemic left and right ventricular developed pressure was expressed as a percentage of its pre-drug, pre-ischemic value.

Assessment of ventricular function. Left and right ventricular function was monitored continuously throughout each experiment as described in Rafiee et al., Circulation 106:239-45 (2002).

Western analysis. Hearts from infant rabbits were isolated and aerobically perfused with bicarbonate buffer for 30 minutes at constant pressure, then perfused with erythropoietin for 5 or 15 minutes. The free wall of the left ventricle was excised and immediately freeze-clamped between stainless steel tongs pre-cooled with liquid nitrogen. Frozen myocardial tissue samples were powdered in a pre-cooled stainless steel mortar and pestle.

The powdered tissue was then processed to obtain cytosolic and particulate fractions for Western analysis, as described in Raflee et al., Circulation 106:239-45 (2002). Powdered tissue was homogenized in sample buffer (50 mM Tris pH 7.5, 5 mM EDTA, 10 mM EGTA, 10 mM benzamidine, 10 μg/ml pepstatin A, 50 μg/ml PMSE, 10 μg/ml aprotinin, 10 μg/ml leupeptin, and 0.3% β-mercaptoenthanol) on ice for 50 strokes. Nuclei and cellular debris was removed by centrifugation (1000 g at. 4° C. for 15 min). The supernatant was transferred to a new cold 1.5 mL microcentrifuge tube. The cytosolic and particulate portions of total cellular proteins were separated by a 30-minute centrifugation at 45000 g. Protein concentrations were determined by the method of Bradford. Equal amounts of protein were analyzed by SDS-PAGE and Western blotting by using either isoform-specific antibodies for phospho-PKC detection or specific antibodies against phosphorylated and non-phosphorylated p38 MAPK, JNK, and p42/44 MAPK. The blots were developed by ECL. Densitometry was performed on each sample and analyzed with the use of NIH image software. Rafiee et al., Circulation 106:239-45 (2002).

Erythropoietin analysis. Venous blood was withdrawn from normoxic and chronically hypoxic infant rabbits (n=5/group). The serum was analyzed for erythropoietin concentration using a standard immunochemiluminometric assay (Quest Diagnostics, San Juan Capistrano, Calif.).

Statistical analysis. Data reported are mean±SD. Statistical analysis was performed by use of repeated measures ANOVA with the Greenhouse-Geisser adjustment used to correct for the inflated risk of a Type I error (Baker et al., Circulation 99:1249-54 (1999)). If significant, the Mann-Whitney test was used as a second step to identify which groups were significantly different. After ANOVA the data were analyzed for differences related to multiple comparisons (Baker et al., Circulation 99:1249-54 (1999)). Significance was set at P<0.05.

Studies and Results

A. Erythropoietin concentration-response studies. Erythropoietin protects the brain against ischemic damage by a mechanism involving protein kinase signaling. Siren, A. L. et al., Proc. Natl. Acad. Sci. USA 98:4044-9 (2001). These pathways also protect the heart against ischemic damage. Rafiee et al., Circulation 106:239-45 (2002). This study was designed to determine whether erythropoietin would also confer cardioprotection.

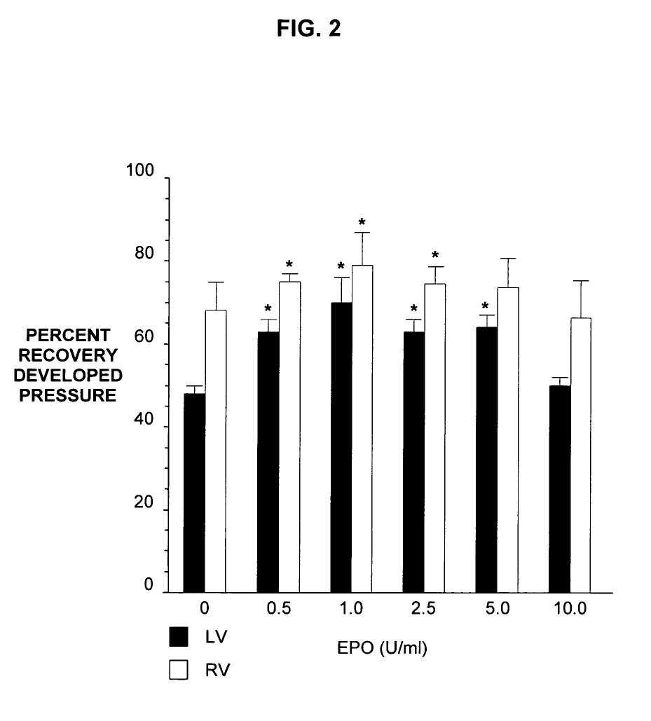

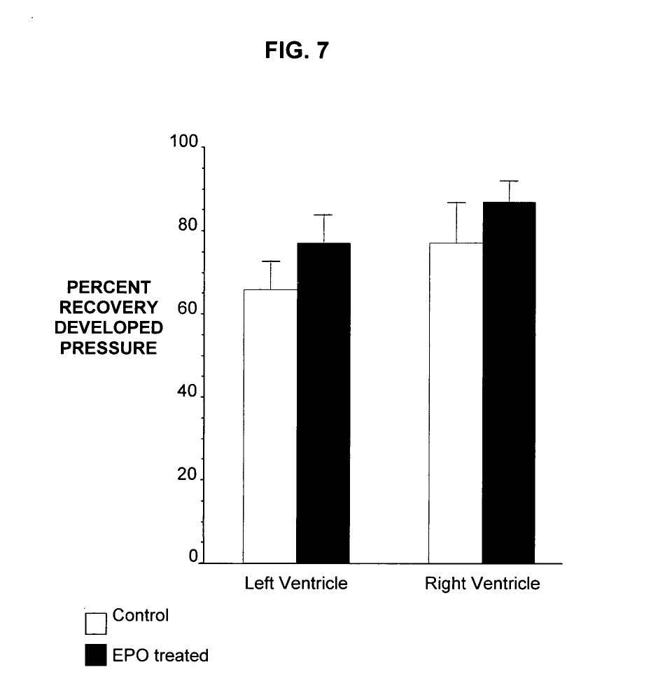

Hearts from New Zealand White rabbits at 9 days of age were perfused with erythropoietin at 0, 0.5, 1.0, 2.5, 5.0, and 10.0 U/ml for 15 minutes prior to 30 minutes global ischemia and 35 minutes reperfusion. Erythropoietin (1.0 U/ml) reduced coronary flow rate prior to ischemia from 7 ml/min/g to 6 ml/min/g but had no effect on heart rate (220±18 beats/min) or developed pressure in left (106±3 mm Hg) or right (42±3 mm Hg) ventricle. Table 1 (below) shows hemodynamic values for erythropoietin concentration-response studies in normoxic hearts and cardioprotection studies in chronically hypoxic hearts (see FIGS. 2 and 7).

| | PRE DRUG | | | | POST DRUG | |
|---|---|---|---|---|---|---|
| | | | Left | Right | | |
| | | | ventricle | ventricle | | |
| | | Coronary | developed | developed | | Coronary |
| | Heart rate | flow rate | pressure | pressure | Heart rate | flow rate |
| Groups | (beats/min) | (ml/min/g) | (mmHg) | (mmHg) | (beats/min) | (ml/min/g) |
| 1. Normoxia | 244 ± 19 | 5 ± 1 | 105 ± 9 | 42 ± 7 | | |
| 2. N + EPO | 223 ± 15 | 6 ± 1 | 106 ± 6 | 41 ± 4 | 196 ± 21 | 3 ± 1 |
| (0.5 U/ml) | | | | | | |
| 3. N + EPO | 229 ± 16 | 6 ± 1 | 101 ± 3 | 40 ± 3 | 199 ± 25 | 3 ± 1 |
| (1.0 U/ml) | | | | | | |
| 4. N + EPO | 226 ± 14 | 6 ± 1 | 101 ± 3 | 42 ± 4 | 210 ± 20 | 4 ± 1 |
| (2.5 U/ml) | | | | | | |
| 5. N + EPO | 236 ± 23 | 6 ± 1 | 101 ± 3 | 43 ± 6 | 221 ± 20 | 4 ± 1 |
| (5.0 U/ml) | | | | | | |
| 6. N + EPO | 248 ± 37 | 6 ± 1 | 103 ± 3 | 42 ± 3 | 232 ± 52 | 5 ± 2 |
| (10 U/ml) | | | | | | |
| 7. Hypoxia | 194 ± 10 | 6 ± 1 | 100 ± 8 | 49 ± 7 | | |
| 8. Hypoxia + EPO | 198 ± 19 | 6 ± 1 | 105 ± 10 | 48 ± 7 | 181 ± 16 | 5 ± 1 |
| (1.0 U/ml) | | | | | | |

| | POST DRUG | | REPERFUSION (35 min) | | | |
|---|---|---|---|---|---|---|
| | Left | Right | | | Left | Right |
| | ventricle | ventricle | | | ventricle | ventricle |
| | developed | developed | | Coronary | developed | developed |
| | pressure | pressure | Heart rate | flow rate | pressure | pressure |
| Groups | (mmHg) | (mmHg) | (beats/min) | (ml/min/g) | (mmHg) | (mmHg) |
| 1. Normoxia | | | 245 ± 23 | 4 ± 1 | 52 ± 4 | 28 ± 4 |
| 2. N + EPO | 102 ± 8 | 38 ± 5 | 210 ± 19 | 4 ± 1 | 67 ± 3 | 31 ± 4 |
| (0.5 U/ml) | | | | | | |
| 3. N + EPO | 99 ± 4 | 38 ± 3 | 210 ± 30 | 5 ± 1 | 71 ± 8 | 31 ± 2 |
| (1.0 U/ml) | | | | | | |
| 4. N + EPO | 103 ± 4 | 41 ± 5 | 224 ± 14 | 5 ± 2 | 64 ± 3 | 31 ± 3 |
| (2.5 U/ml) | | | | | | |
| 5. N + EPO | 102 ± 6 | 43 ± 7 | 226 ± 26 | 4 ± 1 | 64 ± 4 | 32 ± 5 |
| (5.0 U/ml) | | | | | | |
| 6. N + EPO | 101 ± 5 | 40 ± 5 | 230 ± 49 | 5 ± 1 | 52 ± 3 | 28 ± 4 |
| (10 U/ml) | | | | | | |
| 7. Hypoxia | | | 207 ± 29 | 5 ± 1 | 65 ± 4 | 38 ± 6 |
| 8. Hypoxia + EPO | 117 ± 15 | 46 ± 8 | 204 ± 25 | 6 ± 2 | 77 ± 7 | 42 ± 6 |
| (1.0 U/ml) | | | | | | |

EPO = erythropoietin

Erythropoietin increased recovery of left and right ventricular developed pressure following ischemia and reperfusion in a bell-shaped concentration-dependant manner. The optimal concentration that afforded maximal recovery of post-ischemic left and right ventricular developed pressure was manifested at 1.0 U/ml (FIG. 2).

Recovery of coronary flow rate was also increased from 75±2% in untreated hearts to 86±2% of pre-ischemic values in hearts treated with 1.0 U/ml erythropoietin. Recovery of heart rate was unaffected by erythropoietin.

To determine the time-dependency of cardioprotection, hearts were perfused for 5 minutes with erythropoietin prior to ischemia. Treatment of hearts for 5 minutes with erythropoietin at the optimal dose of 1.0 U/ml prior to ischemia did not result in cardioprotection and had no effect on recovery of post-ischemic left ventricular developed pressure (52±6%) compared with untreated controls (49±2%).

These data indicated erythropoietin immediately protects the heart against ischemic injury in a concentration- and time-dependent manner.

B. Role of protein kinases in erythropoietin-induced cardioprotection. Binding of erythropoietin to the erythropoietin receptor activates protein kinase signaling pathways. This study was designed to identify the downstream pathways that underlie cardioprotection conferred by erythropoietin.

A concentration of erythropoietin (1.0 U/ml) that was found to confer optimal cardioprotection was used. Hearts from normoxic rabbits were perfused with protein kinase inhibitors alone for 15 minutes and then combined with erythropoietin for a further 15 minute period prior to ischemia.

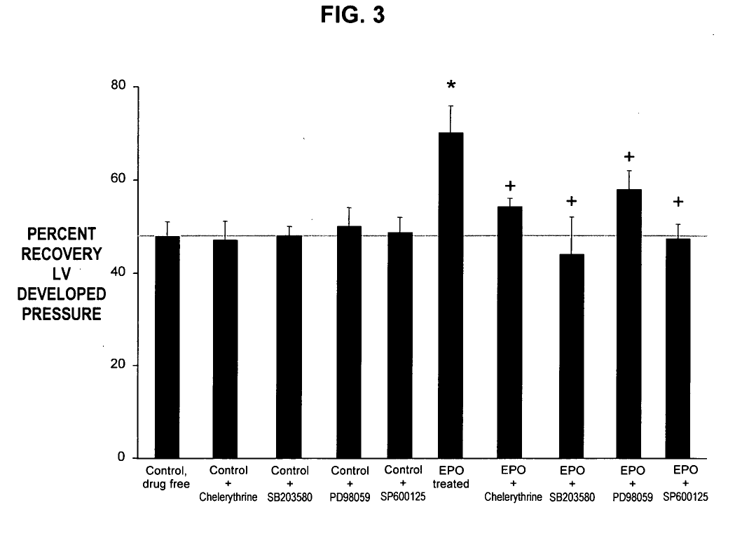

Each of the inhibitors PKC (chelerythrine), p38 MAPK (SB203580), p42/44 MAPK (PD98059) and JNK (SP600125) abolished the cardioprotective effect of erythropoietin (FIG. 3). There was no effect of these inhibitors on cardioprotection in control hearts indicating these protein kinases are not active in untreated hearts.

Cardioprotection by erythropoietin is regulated by inhibitors of protein kinases. To determine if erythropoietin treatment of hearts resulted in activation (phosphorylation) of these protein kinases, the following study was conducted.

Cytosolic and particulate fractions from erythropoietin-treated and untreated hearts were prepared. Protein content for PKCε, p38 MAP kinase, p42/44 MAP kinase and JUN kinase was determined by SDS-PAGE and Western blot analysis using monoclonal antibodies specific for PKC_{ε}, phosphorylated p38 MAP kinase (Thr180/Tyr182), p42/44 MAP kinase (Thr202/Tyr204), and JUN kinase (Thr183/Tyr185). Non-phosphorylated antibodies were used to ensure equal loading of proteins.

Analysis of the cytosolic and particulate fraction revealed that in erythropoietin-treated hearts, PKC_{ε} was activated and translocated from the cytosolic to the particulate fraction. Activation of PKC_{ε} occurs as early as 5 minutes after treatment with erythropoietin and remains active for as long as 15 minutes after treatment. Erythropoietin treatment for 5 minutes resulted in phosphorylation of p38 MAP kinase in the cytosolic fraction but not in the particulate fraction. However, the extent of phosphorylation of p38 MAP kinase declined after 15 minutes treatment with erythropoietin.

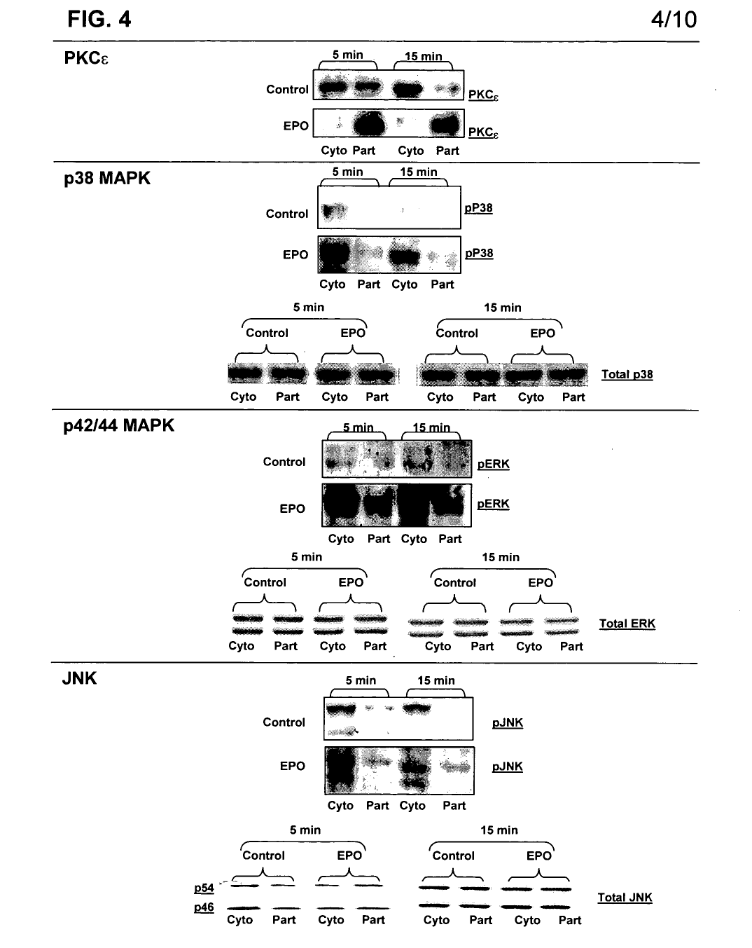

Minimal autophosphorylation of p38 MAP kinase was detected in the cytosolic fraction of untreated hearts. Erythropoietin treatment for 5 minutes resulted in remarkable phosphorylation of p42/44 MAP kinase in the cytosolic fraction with minor phosphorylation in the particulate fraction. In contrast, treatment of hearts with erythropoietin for 15 minutes resulted in enhanced phosphorylation of p42/44 MAP kinase in the cytosolic fraction and in the particulate fraction (FIG. 4). Erythropoietin treatment for 5 minutes also resulted in phosphorylation of JUN kinase in the cytosolic fraction but not in the particulate fraction. Phosphorylation of JUN kinase in the cytosolic fraction declined after 15 minutes of treatment with erythropoietin. Autophosphorylation of JUN kinase (JNK) was present in control hearts not treated with erythropoietin (FIG. 4). This effect is unrelated to mechanical stress caused by inflation of a balloon in the left ventricle, used to measure cavity pressures in the functional recovery studies, as hearts perfused for the kinase studies did not have a balloon inflated in left ventricle (FIG. 4).

Thus, the Western analysis studies confirm the functional recovery studies with inhibitors of protein kinases. The results indicate the immediate cardioprotective effects of erythropoietin are mediated by activation of protein kinase signaling pathways.

C. Role of K_{ATP} channels in erythropoietin-induced cardioprotection. ATP-sensitive K⁺ (K_{ATP}) channels, highly expressed in myocardial sarcolemma and thought to be expressed in myocardial mitochondria, have been found to serve as mediators of cardioprotection. To investigate a role for K_{ATP} channels in mediating erythropoietin-induced cardioprotection, the following study was performed in normoxic rabbits.

Hearts were perfused with K_{ATP} channel blockers alone for 15 minutes and then in combination with erythropoietin (1.0 U/ml) for another 15-minute period prior to ischemia.

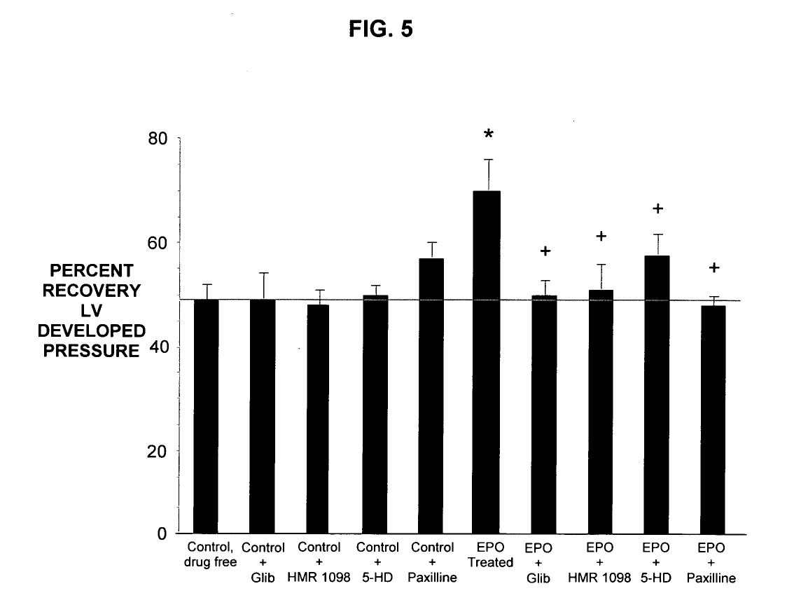

Glibenclamide (3 μM), a non specific K_{ATP} channel blocker, completely abolished the cardioprotective effect of erythropoietin (FIG. 5). The mitochondrial K_{ATP} channel blocker 5-hydroxydecanoate (300 μM) partially and the sarcolemmal K_{ATP} channel blocker HMR 1098 (30 μM) completely blocked the cardioprotective effects of erythropoietin.

Thus, the cardioprotective effects of erythropoietin were shown to be mediated by the sarcolemmal K_{ATP} channel with a possible additional role for the mitochondrial channel.

D. Role of KCa channel in erythropoietin-induced cardioprotection. Another potassium channel, the calcium-activated potassium (KCa) channel located in the inner mitochondrial membrane, has been shown to mediate protection of the heart against ischemia. Xu, W. et al., Science 298:1029-33 (2002). The following study was conducted to determine whether the mitochondrial KCa channel mediates the cardioprotective effects of erythropoietin.

Hearts were perfused with Paxilline (1 μM), a blocker of the KCa channel, alone for 15 minutes and then in combination with erythropoietin (1.0 U/ml) for another 15-minute period prior to ischemia.

Paxilline completely blocked the cardioprotective effect of erythropoietin but had no effect on untreated hearts. The data indicated that the cardioprotective effects of erythropoietin are mediated by the mitochondrial KCa channel (FIG. 5).

E. Role of nitric oxide synthase in erythropoietin-induced cardioprotection. Increased nitric oxide production from nitric oxide synthase serves to protect the heart against ischemic injury. Nitric oxide synthase has also been reported to mediate the cellular effect of erythropoietin. Walker, et al., Am. J. Physiol. Heart Circ. Physiol. 279:H2382-9 (2000). The following study was performed to determine if inhibition of nitric oxide synthase would affect cardioprotection induced with erythropoietin.

Hearts from normoxic rabbits were perfused with nitric oxide synthase inhibitors combined with erythropoietin (1.0 U/ml) for 15 minutes prior to ischemia.

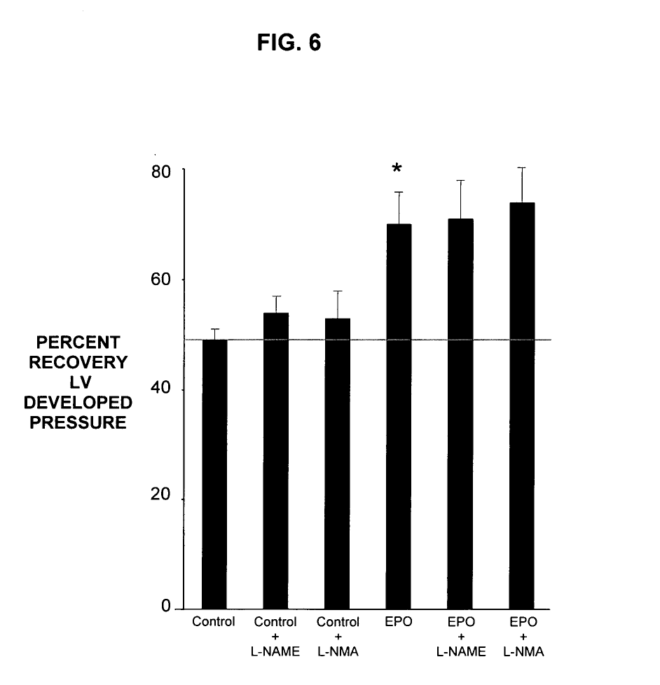

L-NAME (200 μM) or L-NMA (100 μM) did not block the cardioprotective effect of erythropoietin (FIG. 6). Nitrite and nitrate release from hearts before (2.3±0.9 nmoles/min/g) and after (2.4±1.9 nmoles/min/g) 15 minutes treatment with erythropoietin (1.0 U/ml, n=8) were not different. The data indicates that nitric oxide synthase does not play a major role in mediating the cardioprotective effects of erythropoietin in this model.

F. Cardioprotection by erythropoietin in chronically hypoxic hearts. Adaptation to the stress of chronic hypoxia from birth to 10 days of age results in erythropoiesis and also increases resistance to myocardial ischemic injury. Baker, et al., Am. J. Physiol. 268:H1165-1173 (1995). A study was conducted to determine whether erythropoietin confers cardioprotection in chronically hypoxic hearts since many children who have congenital heart defects exhibit varying degrees of cyanosis where erythropoietin increases hematocrit and hemoglobin levels.

Hearts from 10 day old chronically hypoxic rabbits were treated with erythropoietin at a concentration of 1.0 U/ml.

Erythropoietin did not increase recovery of developed pressure in both left and right ventricles (FIG. 7). Thus, the results showed that normoxic and chronically hypoxic hearts respond differently to erythropoietin treatment. Recovery of LVDP was increased by 43% in normoxic infant hearts from 49±2% to 70±6% following treatment with erythropoietin at the optimal dose of 1.0 U/ml. This recovery is comparable with cardioprotection conferred by adaptation to chronic hypoxia. However, erythropoietin treatment did not increase recovery of LVDP and RVDP in hypoxic hearts, suggesting chronically hypoxic hearts are already maximally protected against ischemia.

G. Time to activate protein kinases vs time needed to confer cardioprotection. Phosphorylation of p38 MAP kinase and JUN kinase was maximized following 5 minutes of treatment with erythropoietin, whereas phosphorylation of p42/44 MAP kinase and PKC_{ε} were maximal following 15 minutes of treatment (FIG. 4). Other reports have shown that PKC-MAP kinase pathway is activated within minutes of stimulation and then rapidly declines. Ammargueet al., Biochem. Biophys. Res. Commun. 284:1031-8 (2001); Devemy et al., C. Cell Signal 9:41-6 (1997).

These findings were consistent with the previous observations. Treatment of hearts for 5 minutes with erythropoietin at the optimal dose of 1.0 U/ml prior to ischemia increased the recovery of right ventricular developed pressure from 68±7% to 82±12%, but has no effect on the recovery of left ventricular developed pressure (52±6% vs. 49±2%). The minimum treatement period with erythropoietin needed to confer cardioprotection in both left and right ventricle was 15 minutes.

Hearts were also treated for 20 minutes with erythropoietin (1.0 U/ml). However, there was no further increase in cardioprotection above that conferred following a 15-minute treatment. A possible explanation for the difference in time to activate protein kinases vs. the time needed to confer cardioprotection is that the cardioprotective effect of activation of either of these protein kinases is likely mediated by a downstream component (for example, potassium channels) but not by the kinase per se. Thus, 5 minutes of treatment with erythropoietin is sufficient to activate protein kinases, but insufficient to trigger subsequent downstream components that confer cardioprotection.

H. Circulating erythropoietin levels in infant rabbits. To compare the level of erythropoietin that confers optimal cardioprotection (1.0 U/ml) with the levels present in the circulation, serum levels of erythropoietin were determined.

Serum levels of erythropoietin in normoxic and chronically hypoxic infant rabbits were 2.1±0.4 mU/ml and 7.7±4.0 mU/ml, respectively.

Discussion

Administration of erythropoietin for 15 minutes immediately prior to ischemia resulted in a concentration-dependent increase in recovery of left and right ventricular developed pressure in rabbit hearts following myocardial ischemia and reperfusion. The optimal concentration of erythropoietin that afforded maximum recovery of developed pressure was manifested at 1.0 U/ml.

Erythropoietin (1.0 U/ml) treatment resulted in phosphorylation of PKC_{ε}, p38 MAP kinase and p42/44 MAP kinase. The cardioprotective effects of erythropoietin were abolished by the protein kinase inhibitors SB203580 (p38 MAP kinase), SP600125 (JUN kinase), PD98059 (p42/44 MAP kinase) and chelerythrine (PKC) as well as the potassium channel blockers glibenclamide, HMR 1098, 5-HD and Paxilline. Nitrite and nitrate release from hearts before (2.3±0.9 nmol/min/g) and after (2.4±0.9 nmol/min/g) 15-minute treatment with erythropoietin (1.0 U/ml) were not different. L-NAME and L-NMA did not block the cardioprotective effect of erythropoietin.

The results demonstrated that acute administration of erythropoietin exerted a concentration-dependent cardioprotective effect in isolated infant rabbit hearts via a mechanism involving activation of protein kinases and potassium channels, but not nitric oxide synthase. The results show that rapid activation of protein kinases by erythropoietin represents an important cardioprotective effect, which is achieved at physiological concentrations.

The studies showed that erythropoietin immediately exerts a concentration- and time-dependent cardioprotective effect by activation of downstream protein kinases (e.g., PKC_{ε}, p38 MAP kinase, and p42/44 MAP kinase) with increased resistance to myocardial ischemia mediated by potassium channels but not nitric oxide synthase. The optimal concentration of 1.0 U/ml needed to confer protection against cardiac ischemia was approximately 100 times above levels present during chronic hypoxia or anemia and 500 times above plasma erythropoietin levels of 0.01-0.03 U/mL present in the circulation of normoxic subjects. Increased resistance to myocardial ischemia was observed immediately after treatment with erythropoietin, indicating that induction of new genes is not necessary for its cardioprotective effect to be manifested. The study demonstrates the biological effects of erythropoietin are mediated by a signal pathway that results in immediate activation of two potassium channels, the K_{ATP} and the KCa channel. Protection by erythropoietin is redundant of the cardioprotective effects of chronic hypoxia. Activation of the p38 MAP kinase pathway is responsible for increased cardioprotection in the chronically hypoxic heart. Rafiee, P. et al., Circulation 106:239-45 (2002). The study shows that erythropoietin induces activation of the MAP kinase pathway in the myocardium and also involves a unique and strong activation of PKC.

The results also show that erythropoietin confers immediate cardioprotection by activating protein kinase signaling pathways and potassium channels (sarcolemmal K_{ATP} and mitochondrial KCa). Several distinct types of potassium channel are present in the heart, of which two the K_{ATP} and the KCa channel serve to protect the heart against conditions of oxygen deprivation, such as hypoxia and ischemia. The results showed that erythropoietin-induced protection against ischemia is completely prevented by glibenclamide, a non-specific K_{ATP} channel blocker and by HMR 1098, a sarcolemmal specific K_{ATP} channel blocker. In contrast, 5-HD, a blocker of the mitochondrial K_{ATP} channel only partially prevented the cardioprotective effects of erythropoietin. Furthermore, paxilline, a blocker of both sarcolemmal and mitochondrial KCa channels completely abrogated the protection provided by erythropoietin. The sarcolemmal K_{ATP} channel and the sarcolemmal and mitochondrial KCa channels appear to play a pivotal role with a partial involvement of the mitochondrial K_{ATP} channel in erythropoietin-induced cardioprotection. G. J. Gross, Basic Res. Cardiol. 95:280-284 (2000); Sato et al., Basic Res. Cardiol. 95:285-289 (2000). These potassium channels are thought to be located at two sites within the cell, the sarcolemma and the mitochondria. Once activated, sarcolemmal K_{ATP} and KCa channels promote potassium efflux from the cytosol to outside the cell, while activation of mitochondrial K_{ATP} and KCa channels result in an influx of potassium from the cytosol into the mitochondria. Activation of sarcolemmal K_{ATP} and KCa channels may act to reduce calcium influx into the cell during ischemia. In addition, the sarcolemmal K_{ATP} channel may also be responsible for opening the mitochondrial K_{ATP} channel. In contrast, activation of mitochondrial K_{ATP} and KCa channels may mediate cardioprotection by improved energetics (Eells et al., Circ. Res. 87:915-921 (2000); Xu et al., Science 298:1029-1033 (2002)).

The role of protein kinases and potassium channels in the signal transduction pathway by which erythropoietin increases the resistance of the infant heart to ischemia is based on experiments with kinase inhibitors and potassium channel blockers applied at conventional inhibitory concentrations. This pharmacological approach is dependent on the relative specificity of the inhibitors and blockers used. For example, the role of the sarcolemmal K_{ATP} channel in erythropoietin-induced cardioprotection is based on pharmacological studies with HMR 1098, a blocker of this channel. The specificity of this blocker has recently been questioned as HMR 1098 abolishes the cardioprotective effect of diazoxide, an opener of the mitochondrial K_{ATP} channel (Suzuki et al., Circulation 107:682-685 (2003)). However, HMR 1098 has no effect on the activity of reconstituted mitochondrial K_{ATP} channels (Zhang et al., Circ. Res. 89:1177-1183 (2001)). The cardioprotective effect of erythropoietin is due in part to activation of mitochondria KCa channels located in the cardiomyocytes (Eells et al., Circ. Res. 87:915-921 (2000); Xu et al., Science 298:1029-1033 (2002)). However, this channel may exist in other locations in the heart such as the sarcolemma and may exert its effect on other KCa channels such as those present in the cardiac nerves and smooth muscle cells.

The above study indicates that erythropoietin confers cardioprotection by a mechanism that does not appear to involve nitric oxide synthase. This finding contrasts with other studies where erythropoietin (20 U/ml) stimulates nitric oxide release from endothelial cells (Wu et al., Clin. Sci. (Lond) 97:413-419 (1999)). Comparison of the experimental protocol between the two studies reveals that chronic treatment with high concentrations of erythropoietin were needed to stimulate nitric oxide release, whereas in the above study, the optimal concentration of erythropoietin is far lower and does not result in erythropoiesis. Thus, the studies demonstrate a novel non-erythropoietic action of erythropoietin that is manifested immediately at pharmacologic levels.

The level of cardioprotection achieved with erythropoietin is comparable to that conferred by ischemic preconditioning. Baker, et al., Circulation 99:1249-54 (1999). Ischemic preconditioning is a powerful endogenous phenomenon in which brief episodes of a subtoxic ischemic insult induces robust protection against more prolonged, lethal ischemia. The molecular mechanisms underlying ischemic preconditioning are still being elucidated and clinical application of ischemic preconditioning remains elusive and has not yet gained widespread acceptance as a treatment strategy.

Pharmacologic preconditioning against ischemia could offer a more practical way of harnessing the molecular mechanisms responsible for increased cardioprotection. The studies show that pharmacological preconditioning through erythropoietin is effective and represents a novel cardioprotective strategy in the setting of elective myocardial ischemia as encountered during cardiac surgery and angioplasty. Advantageously, erythropoietin is currently approved and available for human clinical use. This well-tolerated compound does not require an elaborate drug delivery system as is needed for many gene-based therapies.

Erythropoietin has been proposed as a mediator of ischemic preconditioning in the brain since it is produced after lethal ischemic or hypoxic insults. Digicaylioglu, et al., Nature 412:641-7 (2001); Siebenlist, U., Nature 412:601-2. (2001). However, this function has not previously been demonstrated in the heart or other organs.

The study in infant rabbit myocardium demonstrated that erythropoietin protected adult myocardium against ischemia and provided mechanistic data on signaling pathways associated with cardioprotection by erythropoietin. Neural expression of erythropoietin is actually reduced after stimuli that induce ischemic preconditioning in the brain. Jones, et al., J. Cereb. Blood Flow Metab. 21:1105-14 (2001). In addition, lethal stresses and hypoxia/ischemia clearly induce erythropoietin but sublethal preconditioning stimuli may not be potent enough to produce substantial concentrations of erythropoietin. Ibid.

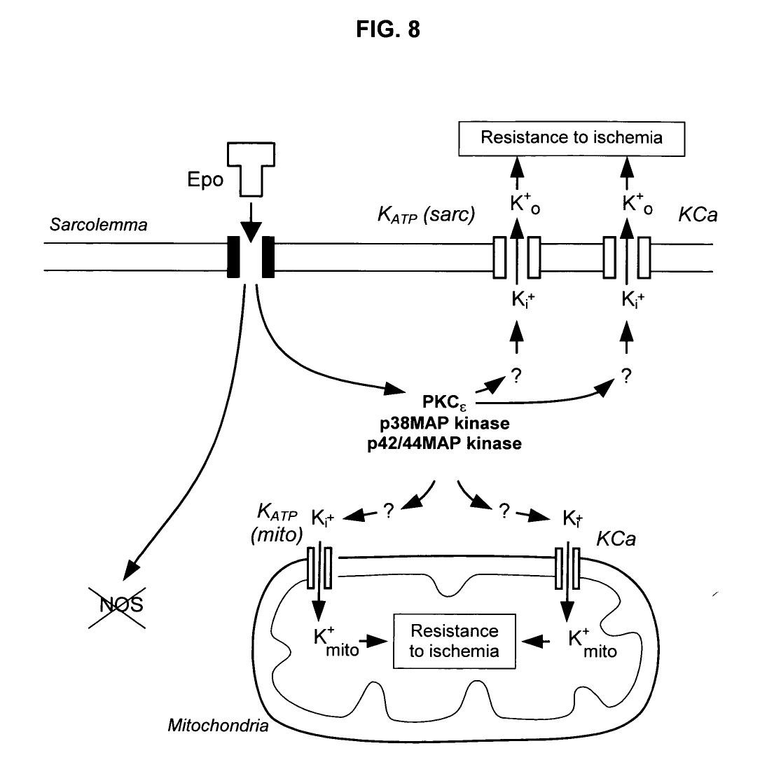

The optimal dose in the study to confer cardioprotection was 1.0 U/ml. In a cerebral model of ischemic injury, for in vivo studies a dose of erythropoietin at 5,000 U/kg conferred protection (Siren et al., Proc. Natl. Acad. Sci. USA 98:4044-4049 (2001)). In the above in vitro studies, erythropoietin at 0.5-5.0 units/ml was protective. Erythropoietin (5,000 U/kg) has been used to confer delayed cardioprotection in the rat and rabbit increasing post-ischemic function recovery [16] and decreasing apoptosis [5] and by reducing infarct size [16]. (Parsa et al., J. Clin. Invest. 112:999-1007 (2003); Cai et al., Proc. Natl. Acad. Sci. USA 100:4802-4806 (2003). Thus, the concentration of erythropoietin required to confer protection against ischemia is comparable for both brain and heart. This study provides mechanistic information on protein kinase signal transduction pathways and potassium channels mediating cardioprotection by erythropoietin. A representation of the signaling pathway by which erythropoietin may confer immediate cardioprotection is depicted in FIG. 8.

Erythropoietin was shown to increase resistance to ischemia in normoxic hearts. Thus, a great benefit would be to normoxic infants undergoing cardiac surgery for repair of congenital heart defects. In contrast, hearts adapted to severe chronic hypoxia already exhibit increased resistance to ischemia compared with normoxic hearts. (Baker et al., Am. J. Physiol. 268:H1165-1173 (1995)). The study showed that erythropoietin does not further increase the level of cardioprotection in chronically hypoxic hearts, indicating cardioprotection by erythropoietin is redundant in these hearts. Furthermore, erythropoietin did not appear to exert any adverse effect on ischemic myocardium in chronically hypoxic infants. In human infants with cyanotic heart defects, chronic hypoxia may be intermittent or continuous in nature with the myocardium exposed to varying degrees of hypoxia. Thus, administration of erythropoietin would also confer cardioprotection in infants with mild degrees of hypoxia.

The results demonstrate that erythropoietin is a suitable exogenous agent to pharmacologically precondition the heart against ischemia. The results further show that to confer cardioprotection, erythropoietin is advantageously given before the ischemic insult, including, for example, planned ischemic events such as cardiac surgery, angioplasty or preservation of donor hearts for transplantation.

### EXAMPLE 2

### In Vitro Studies of Immediate Cardioprotective Effect of Erythropoietin Against Regional Myocardial Ischemia

A coronary artery ligation model was used to demonstrate the immediate protective effect of erythropoietin.

Animals used in this study were adult male Sprague Dawley rats (200-350 g, generally 300 g). Animals were housed under standard conditions and allowed to feed ad lib. The Animal Care and Use Committee of the Medical College of Wisconsin approved all procedures performed in accordance with the regulations adopted by the National Institutes of Health.

A myocardial infarction was produced via the ligation of the left main artery using 6-0 prolene suture (See, e.g., Clements-Jewery et al., Br J Pharmacol. 135:807-815, 2002; Farkas et al., J Cardiovasc Pharmacol. 39,412-424, 2002). Rats were anesthetized with sodium pentobarbital (50 mg/kg i.p.). Heparin was then administered (150 U/kg i.p.) to prevent the formation of a thrombus in the coronary vasculature. The heart was then excised and perfused retrogradely with bicarbonate buffer at a pressure of 80 mmHg. (See e.g., Baker et al., Am J Physiol 278, H1395-H1400, 2000). The perfusate was not recirculated. A compliant saline-filled latex balloon placed in the cavity of the left ventricle and connected to a pressure transducer and physiological recording system (Stoelting) was used to measure cavity pressures. The left main coronary artery was identified and ligated with a 5-0 Prolene suture threaded through a polyethylene tube to act as an occluder. A control group included sham operated hearts in which only a suture was passed around the left main coronary artery was performed.

Hearts were perfused with erythropoietin (1.0 U/ml) in the perfusate for 15 minutes prior to the onset of ischemia. Regional ischemia and reperfusion were induced by tightening the occluder and by releasing it. Hearts were then subjected to 30 minutes regional ischemia followed by 3 hours reperfusion. Recovery of left ventricular developed pressure and infarct size/area at risk at 3 hours reperfusion were used to assess resistance to myocardial ischemia. For characterization of infarction size, hearts were perfused with 10 ml bicarbonate buffer containing triphenyltetrazolium chloride (SIGMA) at 37° C.

The heart was sectioned in 2 mm segments from apex to atrio-ventricular groove in a transverse fashion. Each segment was recorded and placed in formalin. After twenty-four (24) hours, the specimen was digitally photographed in a camera mount to normalize specimen-to-lens distance. Each photograph was then appended to Adobe Photoshop (Adobe™) to measure pixel density of infarcted versus non-infarcted areas. The percentage of infarction of each slide was expressed as a percentage of the entire area of the heart. The sum of all specimen percentages resulted in an overall percentage of infarction in each animal.

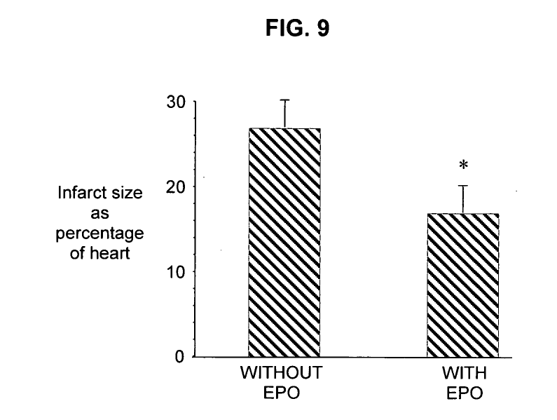

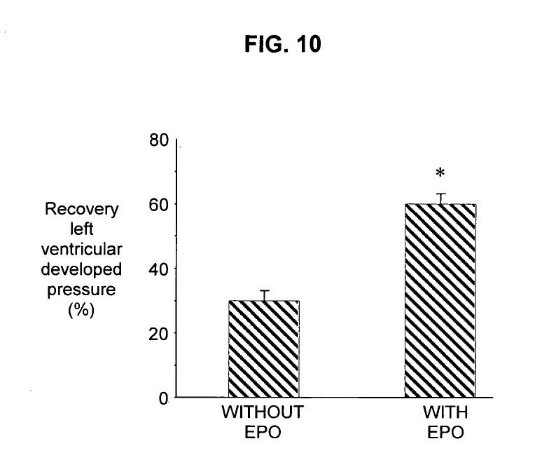

FIG. 9 demonstrates a decrease in myocardial infarct size when erythropoietin was administered 15 minutes prior to regional myocardial ischemia induced by suture ligation of the left main coronary artery. FIG. 10 demonstrates an increase in post-ischemic recovery of left ventricular developed pressure when erythropoietin was administered 15 minutes prior to myocardial ischemia induced by suture ligation of the left main coronary artery.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A method of reducing the effects of myocardial ischemia in a patient, comprising the step of:
administering an effective amount of erythropoietin to the patient in a pharmaceutically acceptable formulation, to provide a substantially immediate decrease in the myocardial ischemia.

2. The method of claim 1, wherein the erythropoietin is administered to a patient.

3. The method of claim 1, wherein a dosage amount of about 50-5,000 U/kg erythropoietin is continuously administered to the patient for about 1-35 minutes to achieve a blood concentration of erythropoietin of about 0.5-10 U/ml.

4. The method of claim 1, wherein the amount of erythropoietin is effective to provide a blood concentration of about 0.5-10 U/ml within about 1-35 minutes following administration.

5. The method of claim 1, wherein the erythropoietin is continuously administered for a period of about 1-20 minutes to achieve a blood concentration of about 0.8-1.5 U/ml.

6. The method of claim 1, wherein the erythropoietin is administered to increase the blood level of erythropoietin in the patient to at least about 100 times above a normal level.

7. The method of claim 6, wherein the erythropoietin is administered to increase the blood level of erythropoietin in the patient to about 0.8-1.5 U/ml.

8. The method of claim 1, wherein the erythropoietin is administered parenterally by intravenous, intramuscular or subcutaneous injection.

9. The method of claim 1, wherein the decrease in the myocardial ischemia is confirmed by at least one of a decrease in tissue necrosis, maintenance of an organ function, a decrease in cardiac enzyme leakage, a decrease in cardiac contractile protein leakage, maintenance of normal left and right cardiac ventricular cavity pressure, volume and flow, a decrease in cardiac arrhythmias, and a decrease in S-T segment elevation.

10. The method of claim 1, wherein the erythropoietin is administered at the commencement of reperfusion, during reperfusion, or both.

11. The method of claim 1, wherein the erythropoietin is administered prior to or during an ischemic event, or both.

12. The method of claim 11, wherein the ischemic event is due to a disease state selected from the group consisting of a myocardial infarction, pulmonary infarction, stroke, and cerebral infarction.

13. The method of claim 11, wherein the ischemic event is due to a disease state selected from the group consisting of peripheral vascular occlusive disease, vascular occlusion, pre-natal or post-natal oxygen deprivation, trauma, chronic obstructive pulmonary disease, emphysema, adult respiratory distress syndrome, septic shock, sickle cell crisis, dysrhythmia, and nitrogen narcosis or neurological deficits caused by a heart-lung bypass procedure.

14. The method of claim 11, wherein the ischemic event comprises a surgical procedure.

15. The method of claim 14, wherein the surgical procedure comprises a heart surgery.

16. The method of claim 11, wherein the ischemic event comprises a heart attack.

17. The method of claim 11, wherein the ischemic event comprises an organ transplant procedure, and the erythropoietin is administered continuously to a donor organ for a period of at least about 15 minutes prior to commencement of the transplant procedure.

18. A method of treating the effects of myocardial ischemia in a patient, comprising the step of: administering an effective amount of erythropoietin to the patient in a pharmaceutically acceptable formulation, wherein a substantially immediate protective effect against myocardial ischemia occurs.

19. A method of reducing the effects of myocardial ischemia in an organ transplant recipient, comprising the step of: exposing the organ to be transplanted to a pharmaceutically acceptable formulation comprising about 0.5-10 U/ml erythropoietin.

20. The method of claim 19, wherein the organ is a heart.

21. The method of claim 19, wherein the step of exposing comprises infusing the formulation into the organ.

22. The method of claim 19, wherein the exposure to erythropoietin is continuous for a period of about 5-30 minutes prior to transplantation.

23. The method of claim 19, wherein the formulation comprises about 0.8-1.5 U/ml erythropoietin.

24. A method of substantially immediately reducing injury associated with myocardial ischemia and reperfusion in a patient, comprising the step of: administering an effective amount of erythropoietin to the patient in a pharmaceutically acceptable formulation to provide a blood concentration of about 0.5-10 U/ml within about 1-35 minutes following administration.

25. A method of preventing or reducing injury associated with myocardial ischemia in a patient, comprising the step of: administering an effective amount of erythropoietin to the patient in a pharmaceutically acceptable formulation for an effective time period to activate a protein kinase to prevent or reduce the ischemic injury.

26. The method of claim 25, wherein the formulation comprises an amount of erythropoietin to provide a blood level of about 0.8-1.5 U/ml erythropoietin within about 1-35 minutes following administration to the patient.

27. A method of preventing or reducing injury associated with myocardial ischemia in a patient, comprising the step of: administering an effective amount of erythropoietin to the patient in a pharmaceutically acceptable formulation for an effective time period to activate a potassium channel to prevent or reduce the ischemic injury.

28. The method of claim 27, wherein the formulation comprises an amount of erythropoietin to provide a blood level of about 0.8-1.5 U/ml erythropoietin within about 1-35 minutes following administration to the patient.

29. A method of providing substantially immediate cardioprotection in a patient, comprising the step of: administering an effective amount of erythropoietin to the patient in a pharmaceutically acceptable formulation, wherein substantially immediate cardioprotection occurs.

30. The method of claim 29, wherein the substantially immediate cardioprotection occurs within about 1-35 minutes of administration of the erythropoietin.

31. The method of claim 30, wherein an amount of erythropoietin is administered over an about 1-35 minute period to provide a blood level of about 0.8-1.5 U/ml erythropoietin.

32. A pharmaceutical composition formulated for the treatment of myocardial ischemia, comprising a therapeutically effective anti-ischemic amount of erythropoietin in a pharmaceutically acceptable vehicle to substantially immediately prevent or reduce myocardial ischemia within about 1-35 minutes of administration.

33. The composition of claim 32, wherein the composition comprises a dose amount of about 50-5,000 U/kg erythropoietin.

34. The composition of claim 32, wherein the composition comprises an amount of erythropoietin to increase the blood level of erythropoietin in a patient to at least about 100 times above a normal level when administered to the patient for a period of about 1-20 minutes.

35. The composition of claim 32, wherein the composition comprises an amount of erythropoietin to increase the blood level of erythropoietin in a patient to about 100-5,000 mU/ml when administered to the patient for a period of about 1-35 minutes.

36. The composition of claim 32, wherein the composition comprises an amount of erythropoietin to activate a protein kinase to prevent or reduce the ischemic injury when administered to the patient for a period of at least about 1-5 minutes.

37. The composition of claim 32, wherein the composition comprises an amount of erythropoietin to activate a potassium channel to prevent or reduce the ischemic injury when administered to the patient for a period of at least about 1-20 minutes.

38. The composition of claim 32, which is formulated for parenteral administration.

39. The composition of claim 32, which is formulated for infusion administration.

40. The composition of claim 32, which is formulated for intranasal administration.

41. The composition of claim 32, which is formulated for transdermal delivery.

42 The composition of claim 32, which is formulated for delivery as a suppository.

43. The composition of claim 32, which is formulated for intraperitoneal delivery.

44. The composition of claim 32, which is formulated for subcutaneous administration.

45. The composition of claim 32, which is formulated for intramuscular administration.

46. A pharmaceutical unit dosage form comprising a therapeutically effective anti-ischemic amount of erythropoietin in a pharmaceutically acceptable vehicle to substantially immediately reduce myocardial ischemia when administered to a patient.
